(19) United States
(12) Patent Application Publication
(10) Pub. No.: US 2024/0177801 A1
(43) Pub. Date: May 30, 2024

(54) METHOD AND USE FOR GENOMIC SELECTION OF NON-FAMILY AND LOW-HERITABILITY VARIETY

(71) Applicant: Yellow Sea Fisheries Research Institute, CAFS, Qingdao (CN)

(72) Inventors: Songlin Chen, Qingdao (CN); Shiyu Qu, Qingdao (CN); Sheng Lu, Qingdao (CN)

(21) Appl. No.: 18/517,041

(22) Filed: Nov. 22, 2023

(30) Foreign Application Priority Data
Nov. 29, 2022 (CN) 202211509983.2

(51) Int. Cl. G16B 20/00; G06N 20/00; G16B 40/20

(52) U.S. Cl. CPC G16B 20/00; G06N 20/00; G16B 40/20

(57) ABSTRACT

A method and system for genomic selection of a non-family and low-heritability variety are provided. The method includes: evaluating a single nucleotide polymorphisms (SNPs) effect size based on a non-equivalent condition of SNPs by correcting a correlation relationship between the SNPs; and breeding the non-family and low-heritability variety based on the SNPs effect size. In the present disclosure, the method for evaluating the SNPs effect size can be used for breeding the non-family and low-heritability variety.

### CROSS REFERENCE TO RELATED APPLICATION

This patent application claims the benefit and priority of Chinese Patent Application No. 202211509983.2 filed on Nov. 29, 2022, the disclosure of which is incorporated by reference herein in its entirety as part of the present application.

### TECHNICAL FIELD

The present disclosure relates to the technical field of genetic breeding, and specifically to a method and use for genomic selection of a non-family and low-heritability variety.

### BACKGROUND

An important basis for food security is the utilization of high-quality germplasm resources. However, the overall seed industry has an olive-shaped distribution, and seeds from the intermediate including corn, soybeans, pigs, beef cattle, mutton sheep and many vegetable varieties are of low quality. Especially in animal husbandry, most of the high-quality breeding sources come from abroad. The lack of in-depth research on the genetic basis of high-quality germplasm has seriously affected the utilization of germplasm resources. Taking rice as an example, in addition to a few sterility genes, disease resistance genes, and insect resistance genes, many genes need to be discovered urgently. Therefore, it is difficult to propose high-quality breeding programs from the gene editing level. Conventional breeding mostly relies on empirical breeding, resulting in a low probability of selection and difficulty in breeding breakthrough varieties.

Currently, computational models for genomic selection are mainly divided into direct methods represented by best linear unbiased prediction (BLUP) and indirect methods represented by Bayes. Both methods assume that single nucleotide polymorphisms (SNPs) are independent of each other and that the effects of SNPs are identically distributed. However, in fact, effective SNPs are generally enriched in quantitative trait locus (QTLs), which shows an obvious correlation. On one hand, the correlation between SNPs may cause estimation errors; on the other hand, the SNPs that are not related to the trait may also dilute an effect of the effective SNPs. At present, models have extremely high requirements on the number of SNPs and the number of reference populations. In order to conduct genomic selection breeding with low-density SNPs, non-equivalence between SNPs and correlation between SNPs are unavoidable problems. Therefore, there is an urgent need for a method for genomic selection of a non-family and low-heritability variety, in which a model for estimating an SNPs effect size is established under the assumption that there is a certain correlation between the SNPs and that the SNPs are non-equivalent.

### SUMMARY

In order to solve the above problems, an objective of the present disclosure is to provide a method for genomic selection of a non-family and low-heritability variety. In the present disclosure, a computational model of whole-genomic selection is established to provide a weak hypothesis model CR-Elastic Net for genomic selection-based breeding. This model solves the problem that existing models of whole-genomic selection breeding have high requirements on the number of SNPs and the number of non-family reference populations, thereby improving an accuracy of animal breeding value estimation through assumptions that are more consistent with a variation pattern of SNPs. In addition, a model that is superior to the advanced level is further provided for non-family and low-heritability populations, thereby effectively estimating a breeding value of the non-family populations using a small number of individuals, and promoting the rapid development of seed industry.

In order to achieve the above technical objective, the present disclosure provides a method for breeding a non-family and low-heritability variety, including:
evaluating a SNPs effect size based on a non-equivalent condition of SNPs by correcting a correlation relationship between the SNPs; and breeding the non-family and low-heritability variety based on the SNPs effect size.

In an embodiment, a process of evaluating the SNPs effect size includes: constructing a repeatable sampling elastic network (CR-Elastic Net) model for evaluating the SNPs effect size according to the non-equivalent condition and the correlation relationship; and evaluating the SNPs effect size by setting a constant fine-tuning penalty and a model cost function.

In an embodiment, during constructing the CR-Elastic Net model, an Elastic Net model is expressed as follows:

$$w = {argmin}_{w} ( {\sum }_{i = 1}^{N} {( y : - {w}^{T} {x}_{i} )}^{2} + λ ρ ‖ w ‖ + \frac{λ ( 1 - ρ )}{2} {‖ w ‖}_{2}^{2} )$$

wherein w represents a matrix of the SNPs effect size; w^{T} represents a transpose of the matrix w; ∥w∥ represents a square root of a maximum characteristic root of a product of a transposed conjugate matrix of w and the matrix w, ∥w∥ |=w^{T}w; ∥w∥₂² represents a square of a Euclidean norm of w, and is a quadratic sum of each of elements in w; yᵢ represents a phenotypic value of the i-th observation; xᵢ represents a genotype of the i-th observation and is a vector of n×1, n represents the number of the SNPs, λ represents the constant fine-tuning penalty, N represents a sample size, and ρ represents a constant of 0 to 1; wherein the model cost function is equivalent to a ridge regression cost function when ρ is 0, and the model cost function is equivalent to a lasso regression cost function when ρ is 1.

In an embodiment, the ρ value representing a correlation between the SNPs is empirically set, and the SNPs are considered independent of each other when the correlation coefficient between the SNPs is less than 1-ρ and the SNPs are considered to be correlated when the correlation coefficient between the SNPs is higher than 1-ρ; and λ with a maximum proportion of a model-explained residual under the ρ value is set through model fitting, and the SNPs effect size is obtained based on ρ and λ.

In an embodiment, during evaluating the SNPs effect size, m subsets with a sample size of n are extracted from an overall sample with replacement through resampling; and the m subsets are subjected to elastic network fitting to obtain m sets of SNPs effect sizes wₖ; wherein distribution of the SNPs obeys original distribution, and a true SNPs effect size converges to a mean of {wₖ} according to a probability.

The present disclosure further provides a use for breeding a non-family and low-heritability variety in a system for breeding the non-family and low-heritability variety, where the system includes:
a first analysis module configured to assume a threshold value of a correlation relationship between SNPs;
a second analysis module configured to acquire an optimal constant fine-tuning penalty under the first analysis module;
an evaluation module configured to evaluate a SNPs effect size; and
a breeding module configured to breed the non-family and low-heritability variety based on the SNPs effect size.

The present disclosure has the following technical effects.

In the present disclosure, the CR-Elastic Net model can be used for breeding a non-family and low-heritability variety, which is beneficial to promoting the development of seed industry.

### BRIEF DESCRIPTION OF THE DRAWINGS

To describe the technical solutions in embodiments of the present disclosure or in the prior art more clearly, the accompanying drawings required for the embodiments are briefly described below. Apparently, the accompanying drawings in the following description show merely some embodiments of the present disclosure, and those of ordinary skill in the art may still derive other accompanying drawings from these accompanying drawings without creative efforts.

FIG. 1 shows a flow chart of a method according to the present disclosure;

FIG. 2 shows a number of traits with stable improvement in a predictive ability of CR-Elastic Net and GBLUP compared to traditional PBLUP among 50 simulated traits under different numbers of reference populations;

FIG. 3 shows a number of traits for which the predictive ability of CR-Elastic Net relative to GBLUP improves by not less than 5%, 10%, and 20% when using different numbers of reference populations among 50 simulated traits;

FIG. 4 shows a comparison of the prediction correlation by averaging cross-validation between CR-Elastic Net and GBLUP in a true data set (Cynoglossus semilaevis); and

FIG. 5 shows a comparison of the prediction correlation by averaging cross-validation between CR-Elastic Net and GBLUP under different numbers of reference populations in a true data set (pig).

### DETAILED DESCRIPTION OF THE EMBODIMENTS

In order to make the objectives, technical solutions and advantages of the embodiments of the present disclosure clearer, the following clearly and completely describes the technical solutions in the embodiments of the present disclosure with reference to the accompanying drawings in the embodiments of the present disclosure. Apparently, the described embodiments are some rather than all of the embodiments of the present disclosure. Generally, the components shown in the accompanying drawings of the embodiments of the present disclosure may be provided and designed in various manners. Therefore, the detailed description of the embodiments of the present disclosure with reference to the accompanying drawings is not intended to limit the protection scope of the present disclosure, but merely to represent the selected embodiments of the present disclosure. All other embodiments obtained by those of ordinary skill in the art based on the embodiments of the present application without creative efforts should fall within the protection scope of the present application.

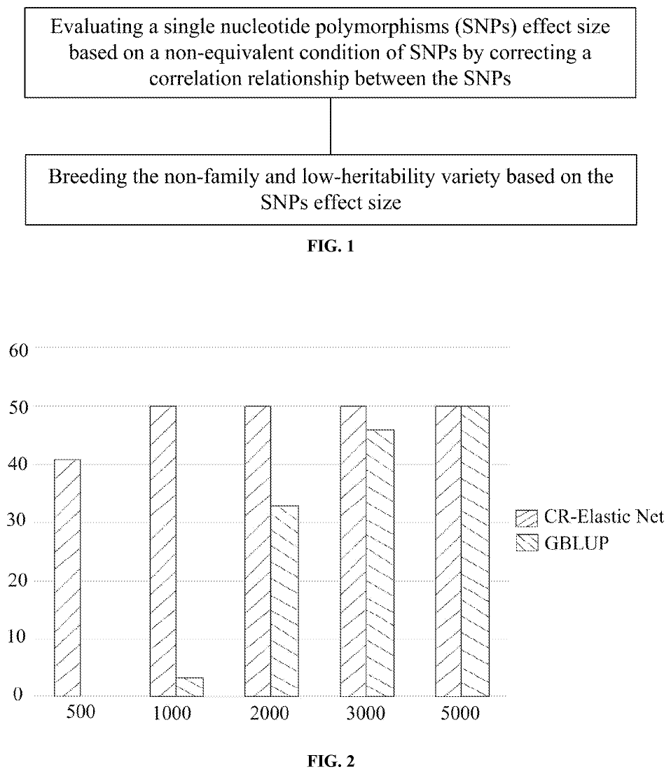

As shown in FIG. 1, the present disclosure provides a method for breeding a non-family and low-heritability variety, including the following steps:
evaluating a SNPs effect size based on a non-equivalent condition of SNPs by correcting a correlation relationship between the SNPs; and breeding the non-family and low-heritability variety based on the SNPs effect size.

In an embodiment, a process of evaluating the SNPs effect size specifically includes: constructing a repeatable sampling elastic network (CR-Elastic Net) model for evaluating the SNPs effect size according to the non-equivalent condition and the correlation relationship; and evaluating the SNPs effect size by setting a constant fine-tuning penalty and a model cost function.

In an embodiment, during constructing the CR-Elastic Net model, an Elastic Net model is expressed as follows:

$$w = {argmin}_{w} ( {\sum }_{i = 1}^{N} {( y : - {w}^{T} {x}_{i} )}^{2} + λ ρ ‖ w ‖ + \frac{λ ( 1 - ρ )}{2} {‖ w ‖}_{2}^{2} )$$

where w represents a matrix of the SNPs effect size; w^{T} represents a transpose of the matrix w; ∥w∥ represents a square root of a maximum characteristic root of a product of a transposed-conjugate matrix of w and the matrix w, ∥w∥=w^{T}w; ∥w∥₂² represents a square of a Euclidean norm of w, and is a quadratic sum of each of elements in w; yᵢ represents a phenotypic value of the i-th observation; xᵢ represents a genotype of the i-th observation and is a vector of n×1, n represents a number of the SNPs, λ represents the constant fine-tuning penalty, N represents a sample size, and ρ represents a constant of 0 to 1; the model cost function is equivalent to a ridge regression cost function when ρ is 0, and the model cost function is equivalent to a lasso regression cost function when ρ is 1. In an embodiment, in the process of setting the constant fine-tuning penalty, the larger the constant fine-tuning penalty, the greater an impact of the L2-regularization penalty term; and when the constant fine-tuning penalty is 0, there is no penalty.

In an embodiment, all SNPs are retained when the model cost function is equivalent to the ridge regression cost function.

In an embodiment, when the model cost function is equivalent to the lasso regression cost function, one of the SNPs is randomly selected if there is a correlation between the SNPs.

In an embodiment, during evaluating the SNPs effect size, m subsets with a sample size of n are extracted from an overall sample with replacement through resampling; the m subsets are subjected to elastic network fitting to obtain m sets of SNPs effect sizes wₖ; and distribution of the SNPs obeys original distribution, and a true SNPs effect size converges to a mean of {wₖ} according to a probability.

The present disclosure further provides a use for breeding a non-family and low-heritability variety in a system for breeding the non-family and low-heritability variety, and the system includes: a first analysis module, a second analysis module, an evaluation module, and a breeding module.

The first analysis module is configured to assume a threshold value of a correlation relationship between SNPs.

The second analysis module is configured to acquire an optimal constant fine-tuning penalty under the first analysis module.

The evaluation module is configured to evaluate a SNPs effect size.

The breeding module is configured to breed the non-family and low-heritability variety based on the SNPs effect size.

The present disclosure further provides a computer program. The computer program implements the method for breeding a non-family and low-heritability variety, and then is embedded into an intelligent device for generating the SNPs effect size under an assumption of the non-equivalent condition of the SNPs and the correlation relationship between the SNPs, and for intelligent breeding of the non-family and low-heritability variety based on the SNPs effect size.

In order to better understand the meaning of the present disclosure, the terms involved in the present disclosure are explained as follows:

Single nucleotide polymorphisms (SNPs) represent DNA sequence polymorphisms caused by single nucleotide variations at the genome level.

Reference population represents a population with phenotypes and genotypes in genomic selection, where the phenotypes are obtained by manual measurement and the genotypes are obtained by genome sequencing. This population is used to train a model.

Candidate population represents a population with only genotypes but no phenotypes in genomic selection. This candidate population includes generally candidate individuals in the breeding. After measuring the genotypes, a breeding value of these individuals can be estimated based on the reference population, and optimal breeding can be conducted based on a level of the breeding value.

Repeatable sampling elastic network (CR-Elastic Net) represents a theory based on resampling theory, elastic network, and minimum mean square error estimation. This method directly estimates the SNPs effect and combines with molecular markers to estimate the breeding value of an individual.

Genomic best linear unbiased prediction (GBLUP) represents a method for estimating the breeding value of an individual by constructing a genomic kinship matrix (G matrix) using high-density molecular markers covering the genome.

Pedigree-based best linear unbiased prediction (PBLUP) represents a method for estimating the breeding value of an individual based on pedigrees.

Genomic estimated breeding value (GEBV) represents a breeding value estimated at the genome level through genomic selection methods.

A true breeding value (TBV) of the simulated data is defined as a sum of the effects of all QTLs on an individual genome.

Minimization criterion of mean-squared error (MSE) represents an optimized error criterion. This criterion minimizes a mean-squared error between unknown quantities and known quantities and then determines the required unknown quantities under this condition.

In the present disclosure, a new computational model (CR-Elastic Net) is used for whole-genomic selection. An example mainly includes the following steps:

### 1) Collection of Simulated Data

The simulated data is 10,000 individuals generated by QMsim2, and 50 traits with an individual heritability of 0.1. There are 5 individual chromosomes, and each of the chromosomes has a length of 100 cM; each chromosome contains 1,000 SNPs and 20 QTLs, and the SNPs and QTLs are randomly distributed on the chromosome; a QTL effect size satisfies the exponential distribution.

### 2) Collection of True Data

### 2.1 Data set of Cynoglossus Semilaevis

Marker loci by Chip 38k SNPs for the Cynoglossus semilaevis are adopted, and there is a resistance phenotype of the Cynoglossus semilaevis to Vibrio harveyi (heritability 0.16). The data set includes: a. Vibrio harveyi resistance phenotypes and genotypes of 1,527 Cynoglossus semilaevis; b. Infection survival rates of 44 Cynoglossus semilaevis and their offspring.

### 2.2 Data set of pig

60k SNPs marker loci of the pig are adopted, and the trait T1 has a heritability of 0.07. The data set includes 3,534 individuals.

### 3) Verification of a Prediction Effect of the Model

All SNPs of simulated data and true data are used to conduct cross-validation with GBLUP and CR-Elastic Net, and a prediction accuracy of the GBLUP and CR-Elastic Net models is separately compared.

In the present disclosure, the CR-Elastic Net can be used to screen high-quality individuals with low heritability, and shows a prediction accuracy significantly improved compared to that of GBLUP. A number of minimum valid reference populations is much lower than that of GBLUP. Therefore, the model of the present disclosure can speed up the breeding of high-quality germplasms.

The present disclosure is described in detail below with reference to the examples.

### Example 1: Generation of Simulated Data and Calculation of Prediction Accuracies of CR-Elastic Net and GBLUP Models (Linux Environment)

### 1. Generation of Simulated Data

Simulated data set had a simulated heritability of 0.1. In an example, a simulated population had 2 generations, each generation had 10,000 individuals, with a male to female ratio of 1:1, and only a first generation had a genotype. There were 100 QTLs and a total of 5 chromosomes, each chromosome was 100 cM in length, and 1,000 SNP sites were randomly distributed on each chromosome. The QTL effect satisfied the exponential distribution. The steps were repeated 50 times and files were generated as: “lm_mrk{1 . . . 50}.txt”; “p1_mrk{1 . . . 50}.txt”; and “p1_data{1 . . . 50}.txt”.

The files were organized into PLINK (map; ped) format through R language:
A resulting output file was converted to .raw (linux) through plink1.9;
A file was obtained as: geno{1 . . . 50}.raw; pheno{1 . . . 50}.csv;
CR-Elastic Net theory and implementation was conducted in julia language;
The organized files included genotype data “geno{1 . . . 50}.raw”, phenotype data “sim{1 . . . 50}pheno.csv”, verification set file “cross.val.txt”, and training set file julia:“cross.train{500;1000;2000}.csv”;
R: Validation set file was: “valname.bin”; and training set collection was: “list_trainnames.bin”;
1.1. A dependency package was imported;
1.2. Calculation was conducted; and
1.3. Model and implementation of GBLUP in R language was conducted:

y=Xb+Za+e
where y represented an n×1 phenotype vector, n represented a sample size; X represented a fixed effect, b represented a vector of the fixed effect; Z as a random effect represented an n×n kinship matrix, e represented a residual, and a represented a vector with a random effect fitting value of n×1. If the data was simulated data, X was a vector of all 1; and if there was a fixed effect, it was a fixed effect matrix.

GBLUP in R language was implemented using an R package “sommer”.

### 2. Determination of the Number of Valid Reference Populations

1,000 individuals were randomly selected from 10,000 genotyped individuals in the simulated data as a validation set, and {500, 1,000, 2,000, 3,000, 4,000, 5,000} individuals were randomly selected from the remaining individuals as a training set. The above steps were repeated 20 times. The GEBVs of training set and validation set were calculated through CR-Elastic Net and GBLUP, respectively, and a Pearson correlation was calculated between the GEBV and TBV (defined as the sum of QTLs) of training set. Since the simulated data was a non-family population, a prediction accuracy of the PBLUP method was 0. With a null hypothesis that a prediction effect of the models (GBLUP; CR-Elastic Net) was inferior to that of BLUP, a sign test was conducted on 50 simulated traits. A confidence level was α=0.05, and Bonferroni was used for correction. As shown in Table 1, CR-Elastic Net and GBLUP effectively improved the prediction accuracy in terms of the number of phenotypes compared to PBLUP in the 50 simulated data sets. A statistical result graph was shown in FIG. 2.

TABLE 1

| Model | 500 | 1000 | 2000 | 3000 | 5000 |
|---|---|---|---|---|---|
| CR-Elastic Net | 41 | 50 | 50 | 50 | 50 |
| GBLUP | 0 | 2 | 33 | 46 | 50 |

It was concluded from Table 1 and FIG. 2 that CR-Elastic Net was stable under a low number of reference population (500), and its prediction accuracy was improved compared to the PBLUP (positive results>80%); while GBLUP was stable when the number of reference populations exceeded 3,000, and its prediction accuracy was improved compared to the PBLUP (positive results>80%).

### 3. Comparison of Data Prediction Accuracy

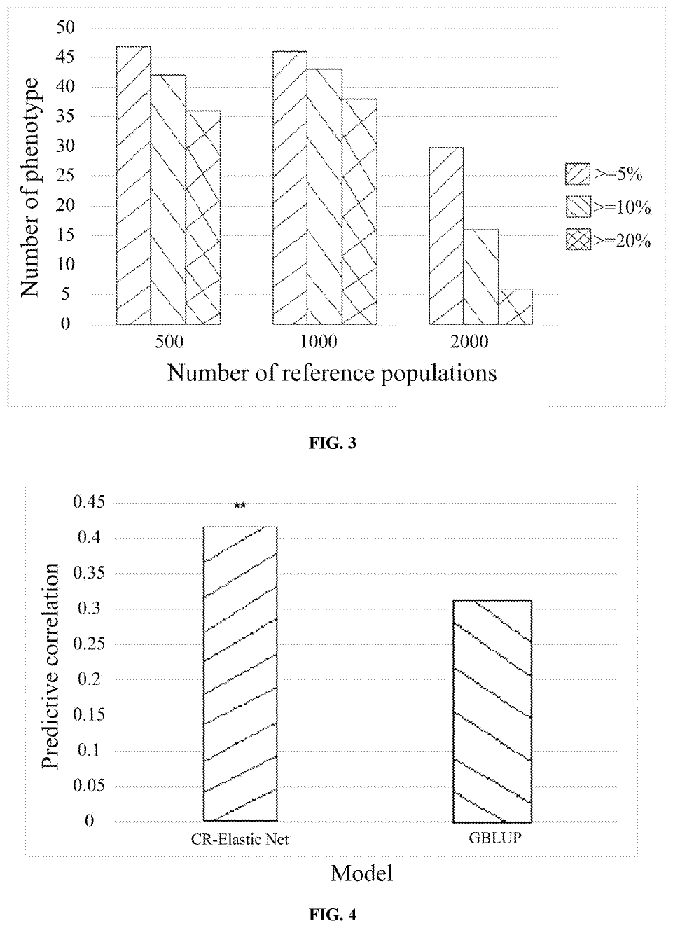

1,000 individuals were randomly selected from 10,000 genotyped individuals in the simulated data as a validation set, and {500, 1,000, 2,000} individuals were randomly selected from the remaining individuals as a training set. The above steps were repeated 20 times. The GEBVs of training set and validation set were calculated through CR-Elastic Net and GBLUP, respectively, and a Pearson correlation was calculated between the GEBV and TBV (defined as the sum of QTLs) of validation set. The prediction accuracy of traits with no prediction effect was defined as 0, and the prediction accuracy was defined as acc=r (GEBV, TBV)/h. The above average accuracy upgrade rate for 50 traits was shown in Table 2, for 50 simulated data sets, the prediction accuracy of CR-Elastic Net was improved relative to that of GBLUP. In the data of Table 2, CR-Elastic Net upgraded by not less than 5%, 10%, and 20% in terms of the number of traits compared to GBLUP under different numbers of reference populations, as shown in FIG. 3.

TABLE 2

| Phenotype | | | |
|---|---|---|---|
| (heritability | | | |
| of 0.1) | 500 | 1000 | 2000 |
| 1 | 0.89392393 | 0.47837174 | 0.202466725 |
| 2 | 0.72213572 | 0.63495987 | 0.04036004 |
| 3 | 0.70106123 | 0.35657483 | 0.035368035 |
| 4 | 0.52158543 | 0.10824315 | 0.012076154 |
| 5 | 0.17938736 | 0.31119025 | 0.075067026 |
| 6 | 0.38561315 | 0.1132377 | 0.035759818 |
| 7 | 0.10903555 | 0.24527933 | 0.001422469 |
| 8 | 0.18886244 | 0.20289291 | 0.166303568 |
| 9 | 0.02390128 | 0.44856583 | 0.199749283 |
| 10 | 0.78442863 | 0.36875949 | 0.014192395 |
| 11 | 1.0191617 | 0.41574107 | 0.30649834 |
| 12 | 0.09580473 | 0.03757701 | 0.018335787 |
| 13 | 0.23033567 | 0.36866774 | 0.069591616 |
| 14 | 0.55996252 | 0.43186511 | 0.096913705 |
| 15 | 0.24584357 | 0.31948676 | 0.117376365 |
| 16 | 0.40232236 | 0.35283525 | 0.085667726 |
| 17 | 0.48602079 | 0.03452012 | −0.015675914 |
| 18 | 0.52385653 | 0.5116557 | 0.127343022 |
| 19 | 0.36045197 | 0.08128424 | 0.073113029 |
| 20 | 0.09294293 | 0.09176114 | −0.049107765 |
| 21 | 0.7683256 | 0.57839532 | 0.111384374 |
| 22 | 0.63300288 | 0.5752662 | 0.048948707 |
| 23 | 0.17916523 | 0.21111961 | 0.061944222 |
| 24 | 0.45734892 | 0.21622776 | 0.325406497 |
| 25 | 0.32684398 | 0.01092117 | 0.035230468 |
| 26 | 0.42691645 | 0.81165282 | 0.054009002 |
| 27 | 0.34896676 | 0.44875963 | 0.058254963 |
| 28 | 0.6317962 | 1.3278451 | 0.144718892 |
| 29 | 0.08532802 | 0.15456114 | 0.003666668 |
| 30 | 0.03735052 | 0.02599409 | −0.013716574 |
| 31 | 0.43951862 | 0.35967577 | 0.073781399 |
| 32 | −0.18888387 | 0.19139473 | 0.100579662 |
| 33 | 0.38855266 | 0.32028469 | −0.033497099 |
| 34 | 0.07453492 | 0.64879127 | 0.096427753 |
| 35 | 1.30815962 | 0.43908449 | 0.097604264 |
| 36 | 1.71265284 | 1.36304731 | 0.291054473 |
| 37 | 0.3807492 | 0.17885202 | 0.029248518 |
| 38 | 0.6154719 | 0.24421971 | −0.020640665 |
| 39 | 0.160401 | 0.09172425 | 0.018661013 |
| 40 | 1.51731426 | 0.25647009 | 0.037148741 |
| 41 | 0.05845484 | 0.22469291 | 0.073023149 |
| 42 | 0.26593459 | 0.537431 | 0.053743717 |
| 43 | 0.60452098 | 0.22200026 | 0.174576277 |
| 44 | 1.08572909 | 0.48199248 | 0.192940989 |
| 45 | 0.26110294 | 0.41441521 | 0.303370961 |
| 46 | 0.52915035 | 0.44747773 | 0.255322796 |
| 47 | 1.01245771 | 0.32206088 | 0.019312053 |
| 48 | 0.61867905 | 0.24304544 | 0.03438679 |
| 49 | 0.31598783 | 0.48759357 | 0.145303626 |
| 50 | 0.19673726 | 0.45888409 | 0.05710474 |
| Average | 0.475578157 | 0.364147 | 0.088842436 |
| Number of | 47 | 46 | 30 |
| Phenotypes ≥0.05 | | | |
| Number of | 42 | 43 | 16 |
| Phenotypes ≥0.1 | | | |
| Number of | 36 | 38 | 6 |
| Phenotype ≥0.2 | | | |

As shown in Table 2 and FIG. 3, with 500, 1,000, and 2,000 individuals as training sets, the CR-Elastic Net model had a prediction effect significantly better than that of GBLUP. Under the 500-individual training set, the prediction effect upgraded by an average of 47.5%, of which prediction accuracies of 42 phenotypes upgraded by not less than 10%, and prediction accuracies of 36 phenotypes upgraded by not less than 20%. Under the 1,000-individual training set, the prediction effect upgraded by an average of 36.41%, of which prediction accuracies of 43 phenotype upgraded by not less than 10%, and prediction accuracies of 38 phenotypes upgraded by not less than 20%. Under the 2,000-individual training set, the prediction effect upgraded by an average of 8.8%, of which prediction accuracies of 16 phenotypes upgraded by not less than 10%, and prediction accuracies of 6 phenotypes upgraded by not less than 20%.

### Example 2: Comparison of Prediction Accuracy of Cynoglossus semilaevis Data set

### 1. Cross-Validation

Genomic selection calculation was conducted on a total of 1,527 Cynoglossus semilaevis individuals from the reference populations and candidate populations in whole-genome resequencing by using the GBLUP algorithm provided by an R language package sommer and combining with the compiled genotype data genotype.csv, phenotype data phonetype.csv, pedigree data pedigree.csv, and cross-validation grouping file K-cross.csv. The GBLUP had a fixed effect, and the CR-Elastic Net converted the fixed effect into a dummy variable. A Pearson correlation between the GEBV and phenotypic value of the validation set was calculated. The accuracies of cross-validation prediction of CR-Elastic Net and GBLUP for 1,572 Cynoglossus semilaevis are showed in Table 3. A paired t test was conducted on the data, and a difference was extremely significant (P=0.001846056; two-tailed). The results were shown in FIG. 4.

TABLE 3

| Cross-validation times | CR-Elastic Net | GBLUP |
|---|---|---|
| 1 | 0.475236763 | 0.311678 |
| 2 | 0.428654087 | 0.326657671 |
| 3 | 0.400412698 | 0.301669515 |
| 4 | 0.422549806 | 0.350388996 |
| 5 | 0.382798822 | 0.269626618 |
| Average | 0.421930435 | 0.312004136 |

### 2. Verification of Offspring Survival Rate

The GEBV of the 44 Cynoglossus semilaevis candidate populations was estimated through CR-Elastic Net and GBLUP separately. After breeding with these individuals, their GEBV and offspring survival rates were shown in Table 4. The table showed the GEBV and infection survival rates (GBLUP and CR-Elastic Net) of the offspring families of 44 Cynoglossus semilaevis candidate individuals.

TABLE 4

| | GBLUP | CR-Elastic Net | Infection survival rate |
|---|---|---|---|
| Family ID | Family GEBV | Family GEBV | (%) |
| 18T01 | 0.23 | 0.568484868 | 78.6 |
| 18T02 | 0.22 | 0.795436787 | 88.4 |
| 18T03 | 0.16 | 0.345814597 | 77.4 |
| 18T04 | 0.11 | 0.056366796 | 61.3 |
| 18T05 | 0.04 | 0.100924937 | 65.2 |
| 18T06 | −0.01 | 0.136970856 | 65.9 |
| 18T08 | −0.01 | 0.302281598 | 65 |
| 18T10 | −0.04 | 0.229883703 | 70 |
| 18T11 | −0.05 | 0.263763038 | 85.7 |
| 18T12 | −0.05 | 0.400912326 | 69.4 |
| 18T13 | −0.09 | 0.003165955 | 64.5 |
| 18T14 | −0.09 | −0.204563213 | 43.5 |
| 18T15 | −0.10 | 0.549133658 | 71.4 |
| 18T16 | −0.14 | −0.037344713 | 53.5 |
| 18T17 | −0.15 | 0.57382091 | 87.5 |
| 18T18 | −0.15 | −0.094325822 | 52.2 |
| 18T21 | −0.16 | 0.266051273 | 44 |
| 18T22 | −0.18 | 0.085093705 | 60.6 |
| 18T23 | −0.20 | 0.332257187 | 58.8 |
| 18T24 | −0.20 | 0.239244795 | 66.7 |
| 18T25 | −0.25 | 0.095658798 | 66.7 |
| 18T26 | −0.27 | −0.124385241 | 40.3 |
| 18T27 | −0.33 | −0.103166247 | 44.8 |

From Table 4, it was calculated that a Pearson correlation coefficient between offspring GEBV and infection survival rate was: CR-Elastic Net (0.7936)>GBLUP (0.71), with an update rate of 12%. This indicated that the CR-Elastic Net model was a relatively accurate prediction model.

### Example 3: Comparison of Prediction Accuracy for Trait T1 With Heritability 0.07 in pig

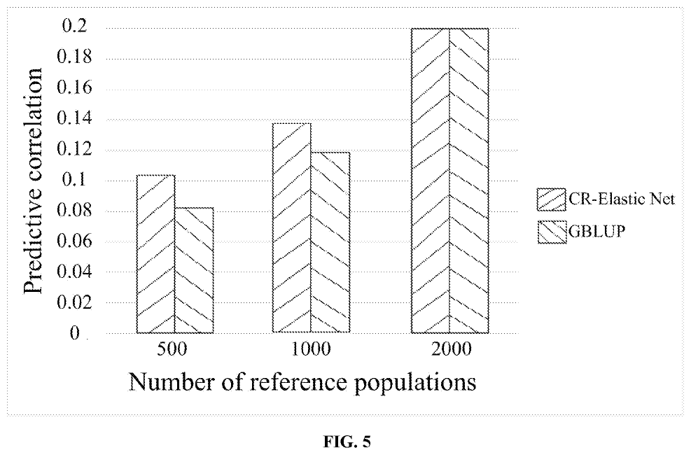

A total of 2,804 pigs with phenotype T1 were selected from a 60 k SNPs data set of 3,534 pigs, from which {500, 1000, 2000} pigs were randomly selected as a training set, while the rest pigs were used as a validation set. The above steps were repeated 50 times. A Pearson correlation between GEBV and phenotype in the validation set was calculated using the CR-Elastic Net and GBLUP models with the training set. The prediction accuracy of traits with no prediction effect was defined as 0. The prediction accuracy was defined as acc=r (GEBV, pheno)/h. An average accuracy and relative upgrade rate of the above 50 repetitions are shown in Table 5. The table shows an accuracy of the dichotomous cross-validation prediction for the trait T1 in pigs with a heritability of 0.07. A comparison of the prediction accuracies of CR-Elastic Net and GBLUP in predicting traits with a heritability of 0.07 under different numbers of reference populations is shown in FIG. 5.

TABLE 5

| | 500 | 1000 | 2000 |
|---|---|---|---|
| CR-Elastic Net | 0.103431287 | 0.138451252 | 0.199294985 |
| GBLUP | 0.08196087 | 0.118309075 | 0.199175451 |
| Upgrade rate | 0.261959355 | 0.170250474 | 0.000600146 |

As shown in Table 5 and FIG. 5, the CR-Elastic Net model also had a significant update compared to that of GBLUP in the true data set of low-heritability phenotypes for pig. This further supported the conclusions drawn from the simulated data.

The results of the above simulated data examples and two true data examples show that the CR-Elastic Net model in the present disclosure has an excellent prediction effect in the breeding of a non-family germplasm with low-heritability traits. For multiple traits of the simulated data, with 500 (average improvement 47.4%), 1,000 (average improvement 36.4%), and 2,000 (average improvement 8.9%) individuals as the training set, the CR-Elastic Net model has a prediction effect significantly better than that of GBLUP, and shows a high proportion of phenotypes with effectively improved prediction accuracy and a relatively low number of effective reference populations required. Under the true data of Cynoglossus semilaevis, the the prediction accuracy by cross-validation has increased by 34%, and the prediction accuracy of offspring is also increased from 0.71 to 0.793, showing an excellent effect. There is also a significant improvement in the validation of low heritability in the pig data set. Two true data sets support the conclusions from the simulated data. To sum up, under the number of commonly used reference populations (500 to 2,000), the CR-Elastic Net model has reached the international top level in a trait prediction effect for a non-family and low-heritability variety, and shows an extremely low requirement on the minimum number of reference populations (>500). As a result, the model can be promoted and applied in subsequent germplasm selection.

The present disclosure is described with reference to the flowcharts and/or block diagrams of the method, the device (system), and the computer program product according to the embodiments of the present disclosure. It should be understood that each flow and/or block in the flowchart and/or block diagram and a combination of the flow and/or block in the flowchart and/or block diagram can be implemented by computer program instructions. These computer program instructions may be provided for a processor of a general-purpose computer, a special-purpose computer, an embedded processor, or other programmable data processing devices to produce a machine, such that instructions executed by the processor of the computer or other programmable data processing devices produce an apparatus used for implementing a function specified in one or more flows of the flowchart and/or one or more blocks of the block diagram.

It should be understood that in the description of the present disclosure, terms such as “first” and “second” are used merely for a descriptive purpose, and should not be construed as indicating or implying relative importance, or implicitly indicating a quantity of indicated technical features. Thus, features defined with “first” and “second” may explicitly or implicitly include one or more of the features. In the description of the present disclosure, “a plurality of” means two or more, unless otherwise specifically defined.

It will be apparent to those skilled in the art that various modifications and variations can be made to the present disclosure without departing from the spirit and scope of the present disclosure. Thus, provided that these modifications and variations of the present disclosure fall within the scope of the claims of the present disclosure and their equivalents, the present disclosure will also be intended to include these modifications and variations.

What is claimed is:

1. A method for genomic selection of a non-family and low-heritability variety, comprising:
evaluating a single nucleotide polymorphisms (SNPs) effect size based on a non-equivalent condition of SNPs by correcting a correlation relationship between the SNPs; and
breeding the non-family and low-heritability variety based on the SNPs effect size.

2. The method according to claim 1, wherein, a process of evaluating the SNPs effect size comprises:
constructing a repeatable sampling elastic network (CR-Elastic Net) model for evaluating the SNPs effect size according to the non-equivalent condition and the correlation relationship; and
acquiring a constant fine-tuning penalty and a model cost function by setting a hyperparameter (an assumed correlation coefficient between the SNPs), and evaluating the SNPs effect size.

3. The method according to claim 2, wherein during constructing the CR-Elastic Net model, an Elastic Net model is expressed as follows:
$$w = {argmin}_{w} ( {\sum }_{i = 1}^{N} {( y : - {w}^{T} {x}_{i} )}^{2} + λ ρ ‖ w ‖ + \frac{λ ( 1 - ρ )}{2} {‖ w ‖}_{2}^{2} )$$
wherein w represents a matrix of the SNPs effect size; w^{T} represents a transpose of the matrix w; ∥w∥ represents a square root of a maximum characteristic root of a product of a transposed conjugate matrix of w and the matrix w, ∥w∥=w^{T}w; ∥w∥₂² represents a square of a Euclidean norm of w, and is a quadratic sum of each of elements in w; yᵢ represents a phenotypic value of an i-th observation; xᵢ represents a genotype of the i-th observation and is a vector of n×1, n represents a number of the SNPs, λ represents the constant fine-tuning penalty, N represents a sample size, and ρ represents a constant of 0 to 1; wherein the model cost function is equivalent to a ridge regression cost function when ρ is 0, and the model cost function is equivalent to a lasso regression cost function when ρ is 1; and a ρ value is empirically set to 0.3, that is, when the correlation coefficient between the SNPs is greater than 0.7 (1-0.3), the SNPs are considered to be correlated.

4. The method according to claim 3, wherein the ρ value representing a correlation between the SNPs is empirically set, and the SNPs are considered independent of each other when the correlation coefficient between the SNPs is less than 1-ρ and the SNPs are considered to be correlated when the correlation coefficient between the SNPs is higher than 1-ρ; and λ with a maximum proportion of a model-explained residual under the ρ value is set through model fitting, and the SNPs effect size is obtained based on ρ and 2.

5. The method according to claim 4, wherein
during evaluating the SNPs effect size, m subsets with a sample size of n are extracted from an overall sample with replacement through resampling; and the m subsets are subjected to elastic network fitting to obtain m sets of SNPs effect sizes wₖ; wherein distribution of the SNPs obeys original distribution, and a true SNPs effect size converges to a mean of {wₖ} according to a probability.

6. A system for breeding the non-family and low-heritability variety, wherein the system comprises:
a first analysis module configured to assume a threshold value of a correlation relationship between SNPs;
a second analysis module configured to acquire an optimal constant fine-tuning penalty under the first analysis module;
an evaluation module configured to evaluate a SNPs effect size; and
a breeding module configured to breed the non-family and low-heritability variety based on the SNPs effect size.
